(19) United States
(12) Patent Application Publication
(10) Pub. No.: US 2026/0123953 A1
(43) Pub. Date: May 7, 2026

(54) Atherectomy-Angioplasty Devices, Systems, and Methods

(71) Applicant: Straub Medical AG, Vilters-Wangs (CH)

(72) Inventors: Danielle Mariah Pompa, Bountiful, UT (US); Bryon Ray Densley, Rochester, MN (US); Hannah Gracie O'neill, Millcreek, UT (US); Carolyn Benner, Karlsruhe (DE); Sebastian Zickwolf, Bretten (DE); Lukas Oettinger, Linkenheim-Hochstetten (DE)

(21) Appl. No.: 18/938,002

(22) Filed: Nov. 5, 2024

(51) Int. Cl. A61B 17/3207; A61B 17/22

(52) U.S. Cl. CPC A61B 17/3207; A61B 17/22012

(57) ABSTRACT

Atherectomy-angioplasty devices, systems, and methods reduce the device costs, procedure times, and risks associated with clinician error and patient trauma that accompany separate atherectomy and angioplasty devices. An example atherectomy-angioplasty device includes a tubular body, an elongate rotor, a plaque-debulking element, and an elongate balloon. The tubular body includes a rotor lumen and an inflation lumen. The elongate rotor is disposed in the rotor lumen. The plaque-debulking element is configured to debulk atherosclerotic or restenotic plaques in a blood-vessel lumen of a patient. To do so, the plaque-debulking element is actuated by the elongate rotor when the elongate rotor is rotated around its longitudinal axis in the rotor lumen. The elongate balloon is over a distal portion of the tubular body. The elongate balloon is fluidly connected to the inflation lumen for inflating or deflating the elongate balloon, as desired, for preventing dispersion of atherosclerotic debris, angioplasty, or both.

### BACKGROUND

Peripheral artery disease (“PAD”) is a condition of reduced or blocked blood flow through the peripheral arteries that currently affects about 202 million people worldwide. In PAD, the peripheral arteries are typically narrowed or blocked as a result of atherosclerosis or the atherosclerotic plaques thereof, which plaques include fat, cholesterol, calcium, and other substances found in the blood. Notably, such plaques can cause the peripheral arteries to stiffen over time, which makes it harder for the heart to pump blood therethrough—even if there is sufficient blood flow through the peripheral arteries.

Atherectomy is indicated for people who have severe atherosclerosis with substantially hardened atherosclerotic plaques. Atherectomy is also indicated for people who have previously had procedures such as angioplasty but have since developed restenotic plaques that reduce or block blood flow. In any case, atherectomy restores patency in people's peripheral arteries by debulking such plaques with any of several atherectomy devices specifically designed therefor. Angioplasty with optional stent placement often follows atherectomy; however, angioplasty is performed with any of several angioplasty devices specifically designed therefor. Thus, while patients often need both atherectomy and angioplasty to restore patency in their peripheral arteries, two different devices are required therefor. This increases device costs, procedure times, risks associated with at least clinician error and patient trauma, or some combination thereof.

Disclosed herein are atherectomy-angioplasty devices, systems, and methods thereof that reduce the foregoing device costs, procedure times, and risks associated with clinician error and patient trauma that accompany separate atherectomy and angioplasty devices.

### SUMMARY

Disclosed herein is an atherectomy-angioplasty device including, in some embodiments, a tubular body, an elongate rotor, a plaque-debulking element, and an elongate balloon. The tubular body includes a rotor lumen and an inflation lumen. The elongate rotor is disposed in the rotor lumen. The plaque-debulking element is configured to debulk atherosclerotic or restenotic plaques in a blood-vessel lumen of a patient. To do so, the plaque-debulking element is actuated by the elongate rotor when the elongate rotor is rotated around its longitudinal axis in the rotor lumen. The elongate balloon is over a distal portion of the tubular body. The elongate balloon is fluidly connected to the inflation lumen for inflating or deflating the elongate balloon as desired, optionally, for angioplasty therewith.

In some embodiments, the elongate balloon is compactly folded in a ready-to-deploy state of the atherectomy-angioplasty device.

In some embodiments, the atherectomy-angioplasty device further includes an expandable stent over the elongate balloon in the ready-to-deploy state of the atherectomy-angioplasty device.

In some embodiments, the atherectomy-angioplasty device further includes a retractable sheath over the elongate balloon in the ready-to-deploy state of the atherectomy-angioplasty device.

In some embodiments, the elongate balloon includes a coating of an antiproliferative drug on an external surface of the elongate balloon. The retractable sheath protects the coating prior to retracting the retractable sheath and inflating the elongate balloon for angioplasty therewith.

In some embodiments, the elongate rotor approximates a shaftless screw auger that creates a vacuum for aspirating atherosclerotic debris into the rotor lumen when the shaftless screw auger is rotated around its longitudinal axis in the rotor lumen.

In some embodiments, the plaque-debulking element is a rotatable cutting tip coupled to the tubular body. The cutting tip is coupled to the shaftless screw auger such that the cutting tip rotates around its longitudinal axis for cutting atherosclerotic or restenotic plaques away from the blood-vessel lumen of the patient when the shaftless screw auger is rotated around its longitudinal axis in the rotor lumen.

In some embodiments, the cutting tip includes at least one side aperture in fluid communication with the rotor lumen. The side aperture allows the atherosclerotic debris to be aspirated into the rotor lumen for extracorporeal collection of the atherosclerotic debris.

In some embodiments, an absence of a shaft in the shaftless screw auger provides a guidewire lumen for a guidewire.

In some embodiments, the elongate rotor approximates a coiled-spring cable.

In some embodiments, the plaque-debulking element is an abrasive mass in a distal portion of the coiled-spring cable. The abrasive mass orbits around a longitudinal axis of the coiled-spring cable for abrading atherosclerotic or restenotic plaques away from the blood-vessel lumen of the patient when the coiled-spring cable is rotated around its longitudinal axis in the rotor lumen.

In some embodiments, the abrasive mass includes an abrasive band around a bump in cable diameter of the coiled-spring cable.

In some embodiments, an axial channel through the coiled-spring cable provides a guidewire lumen for a guidewire.

Also disclosed herein is an atherectomy-angioplasty system including, in some embodiments, atherectomy-angioplasty device and a drive unit operably connected to the atherectomy-angioplasty device. The atherectomy-angioplasty device includes a tubular body, an elongate rotor, a plaque-debulking element, and an elongate balloon. The tubular body includes a rotor lumen and an inflation lumen. The elongate rotor is disposed in the rotor lumen. The plaque-debulking element is configured to debulk atherosclerotic or restenotic plaques in a blood-vessel lumen of a patient. To do so, the plaque-debulking element is actuated by the elongate rotor when the elongate rotor is rotated around its longitudinal axis in the rotor lumen. The elongate balloon is over a distal portion of the tubular body. The elongate balloon is fluidly connected to the inflation lumen for inflating or deflating the elongate balloon as desired, optionally, for angioplasty therewith. The drive unit includes a drive mechanism configured to rotate the elongate rotor around its longitudinal axis in the rotor lumen of the atherectomy-angioplasty device.

In some embodiments, the atherectomy-angioplasty device further includes an expandable stent over a compactly folded elongate balloon in a ready-to-deploy state of the atherectomy-angioplasty device.

In some embodiments, the elongate balloon includes a coating of an antiproliferative drug on an external surface of the elongate balloon.

In some embodiments, the atherectomy-angioplasty system further includes a collection unit fluidly connected to the rotor lumen of the atherectomy-angioplasty device. The collection unit is configured for collecting atherosclerotic debris aspirated into the rotor lumen.

In some embodiments, the atherectomy-angioplasty system further includes a guidewire and a fluid-delivery device. The guidewire is configured for advancing the atherectomy-angioplasty device over the guidewire such that the plaque-debulking element is adjacent the atherosclerotic or restenotic plaques in the blood-vessel lumen of the patient. The fluid-delivery device is configured for inflating or deflating the elongate balloon via the inflation lumen with a fluid.

Also disclosed herein is a method of an atherectomy-angioplasty system including, in some embodiments, obtaining an atherectomy-angioplasty device. The atherectomy-angioplasty device includes a tubular body, an elongate rotor, a plaque-debulking element, and an elongate balloon. The tubular body includes a rotor lumen and an inflation lumen. The elongate rotor is disposed in the rotor lumen. The plaque-debulking element is actuated by the elongate rotor when the elongate rotor is rotated around its longitudinal axis in the rotor lumen. The elongate balloon is over a distal portion of the tubular body. The elongate balloon is fluidly connected to the inflation lumen for inflating or deflating the elongate balloon. The method also includes advancing the plaque-debulking element to a treatment area within a blood-vessel lumen of a patient. The method also includes driving a drive unit operably connected to the atherectomy-angioplasty device. The drive unit includes a drive mechanism configured to rotate the elongate rotor around its longitudinal axis in the rotor lumen of the atherectomy-angioplasty device to debulk an atherosclerotic or restenotic plaque in the treatment area. The method also includes inflating the elongate balloon via the inflation lumen with fluid from a fluid-delivery device such that the elongate balloon is at least in contact with a blood-vessel wall in the treatment area.

In some embodiments, inflating the elongate balloon is performed before driving the drive unit to debulk the atherosclerotic or restenotic plaque in the blood-vessel lumen of the patient. In such embodiments, the elongate balloon blocks blood flow by way of its contact with the blood-vessel wall and, thereby, prevents dispersion of atherosclerotic debris from the treatment area.

In some embodiments, inflating the elongate balloon is performed after driving the drive unit to debulk the atherosclerotic or restenotic plaque in the blood-vessel lumen of the patient. In such embodiments, the elongate balloon effectuates angioplasty in the treatment area.

In some embodiments, the method further includes collecting atherosclerotic debris from the rotor lumen of the atherectomy-angioplasty device with a collection unit fluidly connected to the rotor lumen.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a first atherectomy-angioplasty device with an elongate balloon in an uninflated state in accordance with some embodiments.

FIG. 2 illustrates the first atherectomy-angioplasty device with the elongate balloon in an inflated state in accordance with some embodiments.

FIG. 3 illustrates the first atherectomy-angioplasty device with an expandable stent in an expanded state over the elongate balloon in the inflated state in accordance with some embodiments.

FIG. 4 illustrates a transverse cross-section of the first atherectomy-angioplasty device with an inflation lumen in accordance with some embodiments, the transverse cross-section of the first atherectomy-angioplasty device as indicated in FIG. 1.

FIG. 5 illustrates another transverse cross-section of the first atherectomy-angioplasty device in which the inflation lumen is fluidly connected to the elongate balloon in accordance with some embodiments.

FIG. 6 illustrates a second atherectomy-angioplasty device with the elongate balloon in the uninflated state in accordance with some embodiments.

FIG. 7 illustrates the second atherectomy-angioplasty device with the elongate balloon in the inflated state in accordance with some embodiments.

FIG. 8 illustrates the second atherectomy-angioplasty device with the expandable stent in the expanded state over the elongate balloon in the inflated state in accordance with some embodiments.

FIG. 9 illustrates a transverse cross-section of the second atherectomy-angioplasty device with the inflation lumen in accordance with some embodiments, the transverse cross-section of the second atherectomy-angioplasty device as indicated in FIG. 6.

FIG. 10 illustrates another transverse cross-section of the second atherectomy-angioplasty device in which the inflation lumen is fluidly connected to the elongate balloon in accordance with some embodiments.

FIG. 11 illustrates an atherectomy-angioplasty system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

As set forth above, atherectomy is indicated for people who have severe atherosclerosis with substantially hardened atherosclerotic plaques. Atherectomy is also indicated for people who have previously had procedures such as angioplasty but have since developed restenotic plaques that reduce or block blood flow. In any case, atherectomy restores patency in people's peripheral arteries by debulking such plaques with any of several atherectomy devices specifically designed therefor. Angioplasty with optional stent placement often follows atherectomy; however, angioplasty is performed with any of several angioplasty devices specifically designed therefor. Thus, while patients often need both atherectomy and angioplasty to restore patency in their peripheral arteries, two different devices are required therefor. This increases device costs, procedure times, risks associated with at least clinician error and patient trauma, or some combination thereof.

Disclosed herein are atherectomy-angioplasty devices, systems, and methods thereof that reduce the foregoing device costs, procedure times, and risks associated with clinician error and patient trauma that accompany separate atherectomy and angioplasty devices.

### Devices

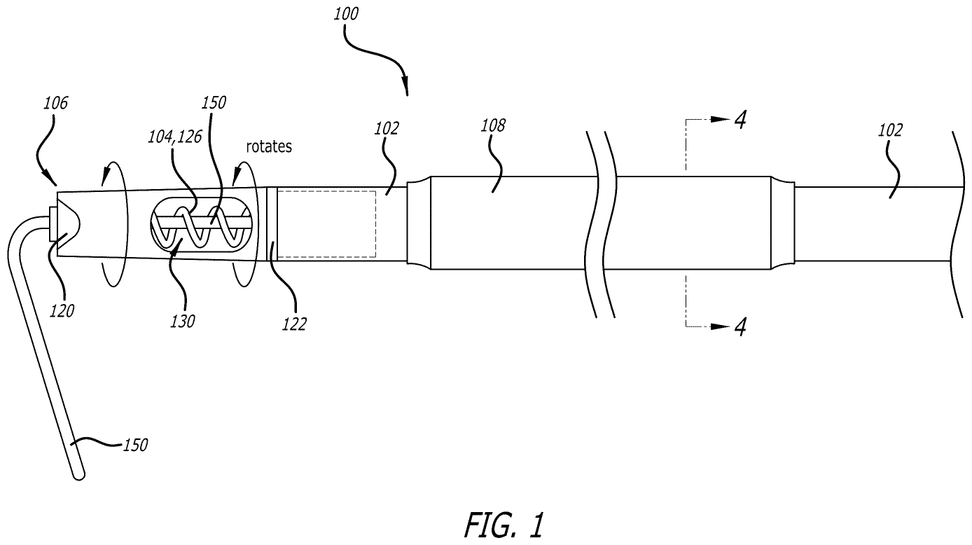

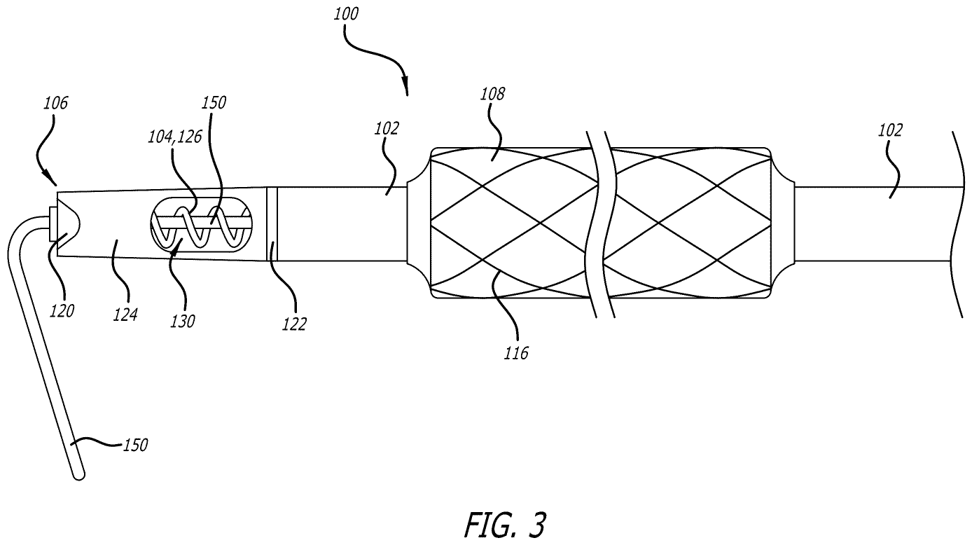

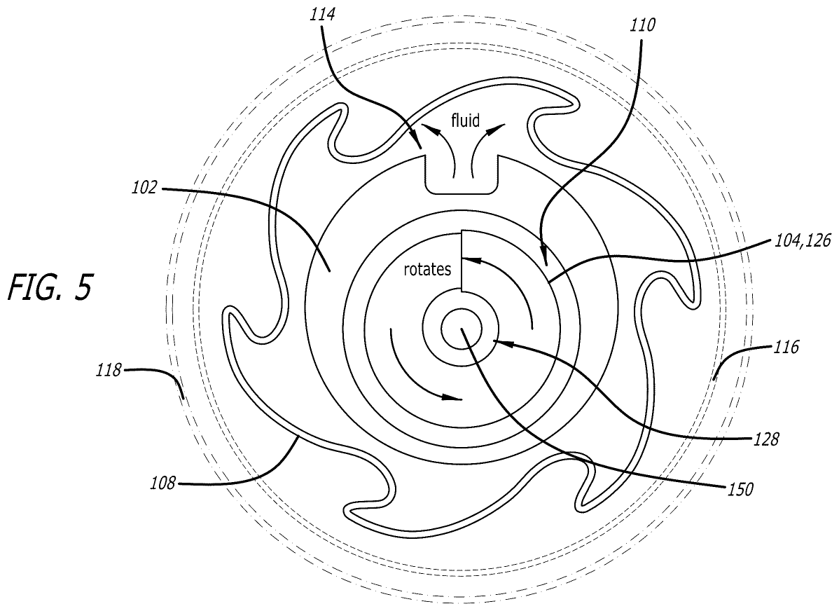

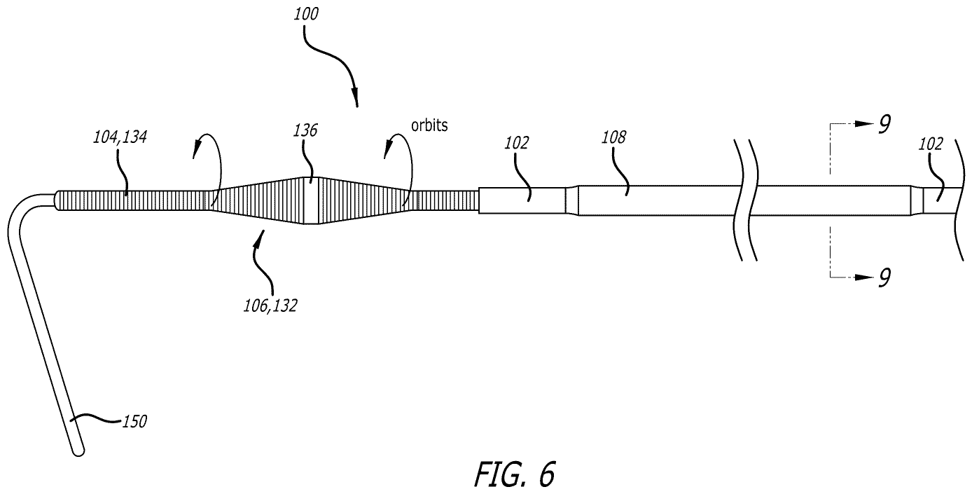

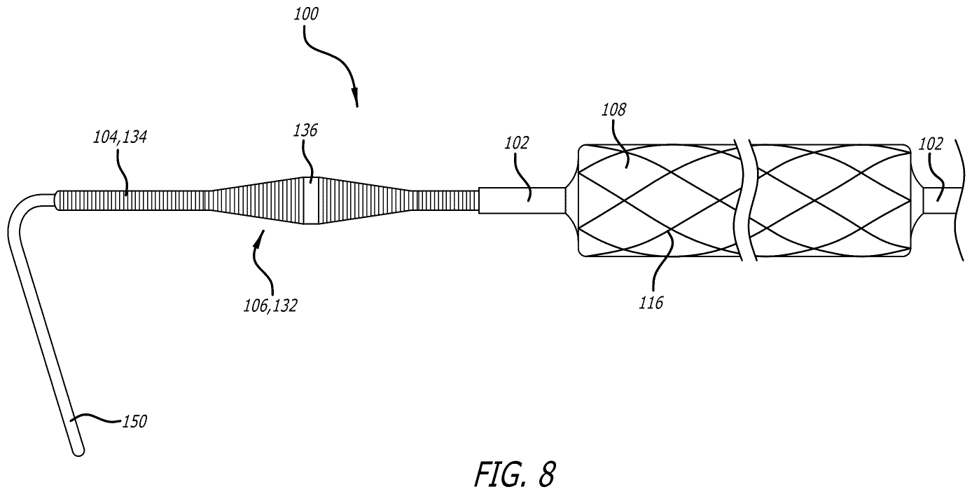

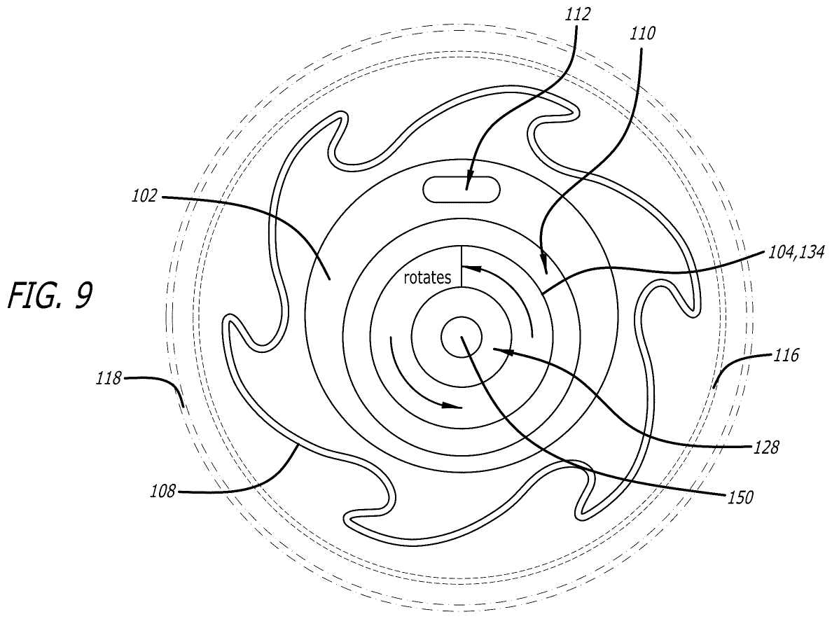

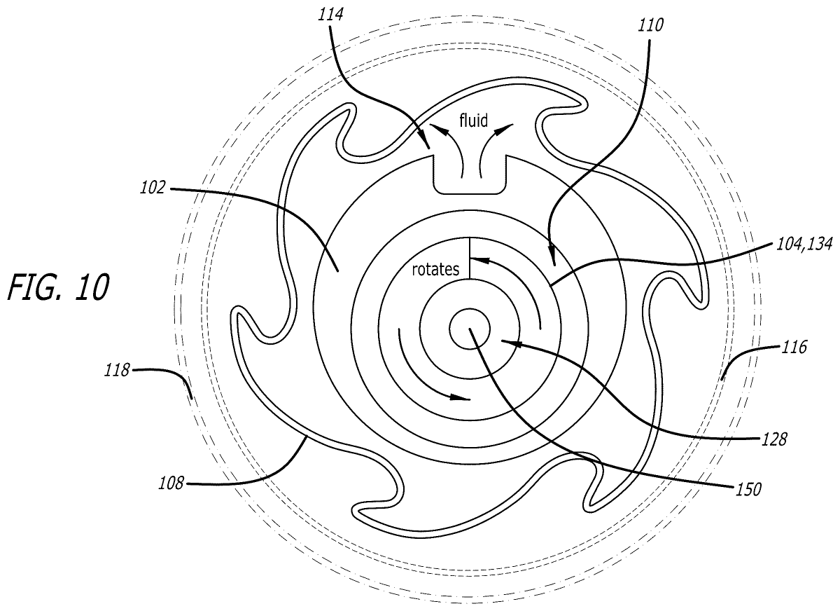

FIGS. 1-5 illustrate an atherectomy-angioplasty device 100 in accordance with some embodiments in which the atherectomy-angioplasty device 100 is a rotary device. FIGS. 6-10 illustrate the atherectomy-angioplasty device 100 in accordance with some other embodiments in which the atherectomy-angioplasty device 100 is an orbital device. It should be understood that while the same reference number “100” is used for the atherectomy-angioplasty device 100 of both groups of the foregoing figures, this is an expository convenience for conveying certain genus-level features of the atherectomy-angioplasty device 100. Species-level features of the atherectomy-angioplasty device 100 are primarily, but not exclusively, described with respect to FIGS. 1-5 for the rotary-device embodiment of the atherectomy-angioplasty device 100 and FIGS. 6-10 for the orbital-device embodiment of the atherectomy-angioplasty device 100.

As shown, the atherectomy-angioplasty device 100 includes a tubular body 102, an elongate rotor 104, a plaque-debulking element 106, and an elongate balloon 108.

The tubular body 102 includes at least a rotor lumen 110 in which the elongate rotor 104 is disposed and an inflation lumen 112 to which the elongate balloon 108 is fluidly connected through an inflation-lumen aperture 114 for inflating or deflating the elongate balloon 108 as desired. Such a tubular body 102 is of a flexible construction for advancing the atherectomy-angioplasty device 100 to a treatment area within a blood-vessel lumen of a patient without patient trauma. Further, the tubular body 102 is of a durable construction for supporting the elongate rotor 104 while it is rotated around its longitudinal axis in the rotor lumen 110 while effectuating atherectomy by way of the plaque-debulking element 106 coupled thereto.

The elongate rotor 104 is rotated around its longitudinal axis in the rotor lumen 110 to actuate the plaque-debulking element 106 coupled thereto for atherectomy. While the elongate rotor 104 varies with respect to whether the atherectomy-angioplasty device 100 is the rotary device of FIGS. 1-5 or the orbital device of FIGS. 6-10, the elongate rotor 104 is of a flexible construction for flexing with the tubular body 102 while the atherectomy-angioplasty device 100 is advanced to the treatment area within the blood-vessel lumen of the patient. Further, the elongate rotor 104 is of a durable construction for transferring torque from the drive mechanism 142 of the drive unit 140 to the plaque-debulking element 106 coupled thereto for effectuating atherectomy.

The plaque-debulking element 106 is configured to debulk atherosclerotic or restenotic plaques in the blood-vessel lumen of the patient by cutting such plaques away from the blood-vessel lumen of the patient, abrading such plaques away from the blood-vessel lumen of the patient, or the like. To do so, the plaque-debulking element 106 is actuated by the elongate rotor 104 when the elongate rotor 104 is rotated around its longitudinal axis in the rotor lumen 110. Whether the plaque-debulking element 106 rotates or orbits around the longitudinal axis of the elongate rotor 104 defines whether the atherectomy-angioplasty device 100 is a rotary or orbital device like that described herein.

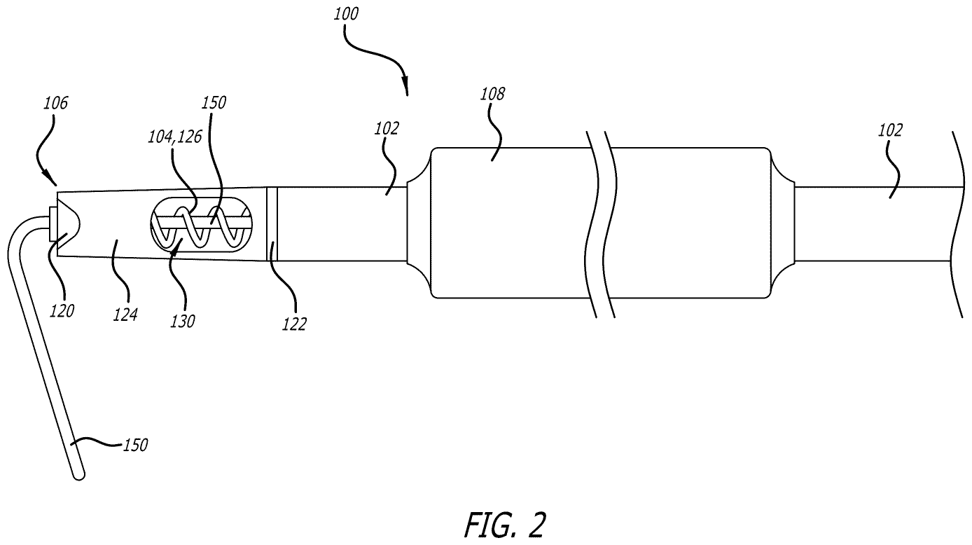

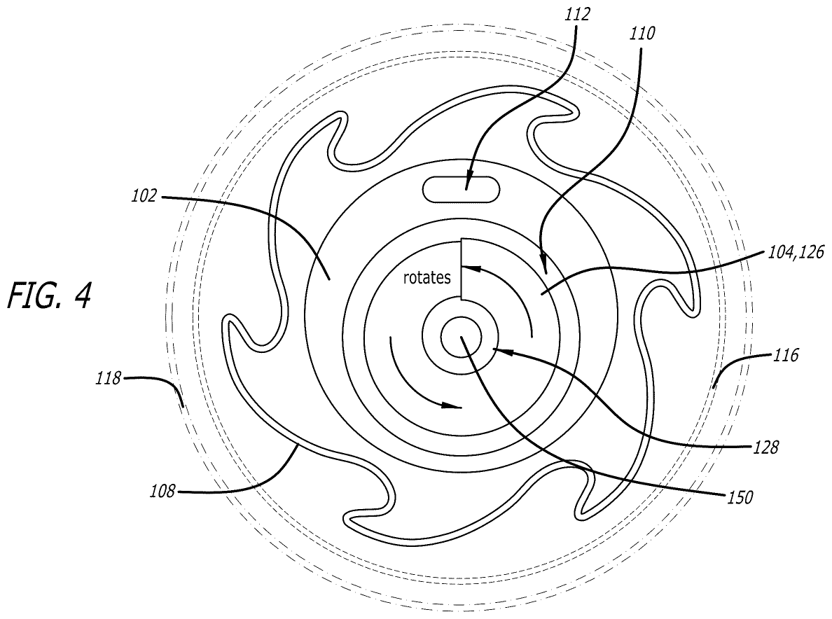

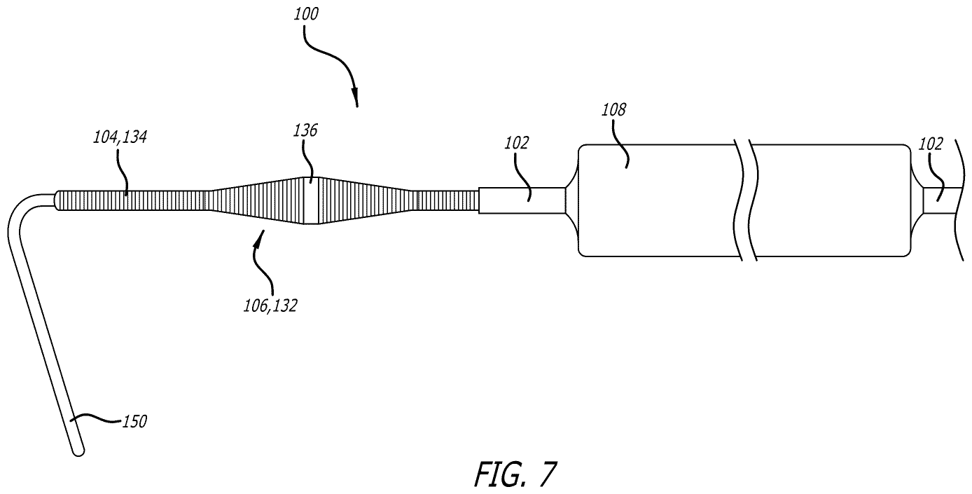

The elongate balloon 108 is over a distal portion of the tubular body 102 but wholly proximal of the plaque-debulking element 106 such that there is no interference between the plaque-debulking element 106 and the elongate balloon 108 during at least atherectomy with the atherectomy-angioplasty device 100. Such an elongate balloon 108 can be about 60-80 mm long, which is sufficient for even relatively long atherosclerotic or restenotic plaques in that the elongate balloon 108 can be inflated and deflated numerous times during angioplasty therewith. As best shown in FIGS. 4, 5, 9, and 10, the elongate balloon 108 is compactly folded in a ready-to-deploy state of the atherectomy-angioplasty device 100 so as to minimize or eliminate patient trauma while advancing the atherectomy-angioplasty device 100 to the treatment area within the blood-vessel lumen of the patient. As best shown in FIGS. 2 and 7, the elongate balloon 108 is in an inflated state subsequent to inflating the elongate balloon 108 via the inflation lumen 112 with a fluid such as saline from a fluid-delivery device such as a syringe fluidly connected to the inflation lumen 112 of the tubular body 102.

As further shown in the FIGS. 4, 5, 9, and 10, the atherectomy-angioplasty device 100 can additionally include an expandable stent 116, a retractable sheath 118, or both over the balloon in the ready-to-deploy state of the atherectomy-angioplasty device 100. Notably, the retractable sheath 118 is useful for covering and protecting a coating of an antiproliferative drug on an external surface of the elongate balloon 108, a same or different coating of such an antiproliferative drug on an external surface of the expandable stent 116, or both prior to retracting the retractable sheath 118. As to retracting the retractable sheath 118, it can be retracted prior to inflating the elongate balloon 108 and, if the expandable stent 116 is present, expanding the expandable stent 116, by way of a retraction mechanism incorporated into the atherectomy-angioplasty device 100. Such a retraction mechanism can include, but is not limited to, a manually operated control element such as a slide button or switch in an extracorporeal portion of the atherectomy-angioplasty device 100. The control element can be operably connected to the retractable sheath 118 by a retraction guidewire 150 in a retraction-guidewire lumen 128 of the tubular body 102 for retracting the retractable sheath 118.

As set forth above, FIGS. 1-5 illustrate the atherectomy-angioplasty device 100 in accordance with some embodiments in which the atherectomy-angioplasty device 100 is a rotary device.

In such embodiments, the plaque-debulking element 106 is a rotatable cutting tip 120 coupled to the tubular body 102. Such a rotatable cutting tip 120 includes a stationary portion 122 inserted into the tubular body 102 and a rotatable portion 124 coupled to the elongate rotor 104 that rotates around its longitudinal axis relative to the stationary portion 122 for cutting atherosclerotic or restenotic plaques away from the blood-vessel lumen of the patient. The elongate rotor 104 in such embodiments approximates a shaftless screw auger 126, which advantageously provides a guidewire lumen 128 for the guidewire 150 along the longitudinal axis of the shaftless screw auger 126 due to an absence of a shaft there. Notably, the rotatable cutting tip 120 includes at least one side aperture 130 in fluid communication with the rotor lumen 110. Advantageously, the shaftless screw auger 126 creates a vacuum in the rotor lumen 110 when the shaftless screw auger 126 is rotated around its longitudinal axis in the rotor lumen 110, the side aperture 130 thereby allowing atherosclerotic debris to be aspirated into the rotor lumen 110 for extracorporeal collection of the atherosclerotic debris in the collection unit 144.

As set forth above, FIGS. 6-10 illustrate the atherectomy-angioplasty device 100 in accordance with some embodiments in which the atherectomy-angioplasty device 100 is an orbital device.

In such embodiments, the plaque-debulking element 106 is an abrasive mass 132 in a distal portion of the elongate rotor 104 that orbits around the longitudinal axis of the elongate rotor 104 for abrading atherosclerotic or restenotic plaques away from the blood-vessel lumen of the patient. The elongate rotor 104 in such embodiments approximates a coiled-spring cable 134, which advantageously provides the guidewire lumen 128 for the guidewire 150 along the longitudinal axis of the coiled-spring cable 134 due to an otherwise empty axial channel there. Further to the plaque-debulking element 106, the abrasive mass 132 is formed from the distal portion of the elongate rotor 104, the abrasive mass 132 including, but not limited to, an abrasive band 136 around a bump in cable diameter of the coiled-spring cable 134. While the orbital device does not include the atherosclerotic-debris aspiration mechanism of the rotary device, the elongate balloon 108 can be advantageously inflated before debulking the atherosclerotic or restenotic plaque in the blood-vessel lumen of the patient to block blood flow by way contact with a blood-vessel wall. The elongate balloon 108 thereby prevents dispersion of atherosclerotic debris from the treatment area as the abrasive mass 132 orbits around the longitudinal axis of the coiled-spring cable 134 and abrades the atherosclerotic or restenotic plaques away from the blood-vessel lumen of the patient in accordance with the coiled-spring cable 134 being rotated around its longitudinal axis in the rotor lumen 110. Notably, in such embodiments, the elongate balloon 108 can be slidably disposed on the tubular body 102 over the inflation-lumen aperture 114, optionally, between proximal and distal stops, thereby allowing longitudinal movement of the tubular body 102 through the elongate balloon 108 for debulking the atherosclerotic or restenotic plaque in the blood-vessel lumen of the patient while also blocking blood flow with the elongate balloon 108.

### Systems

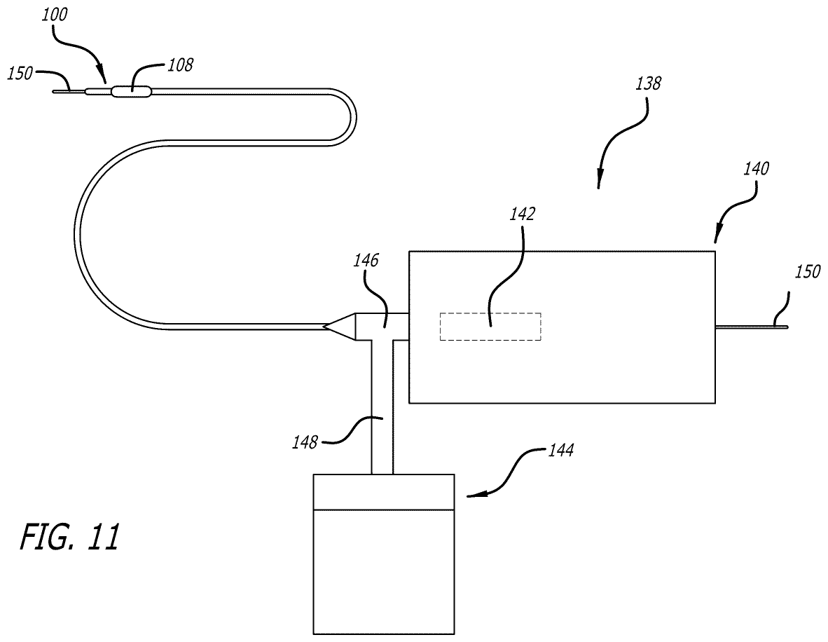

FIG. 11 illustrates an atherectomy-angioplasty system 138 in accordance with some embodiments.

As shown, the atherectomy-angioplasty system 138 includes the atherectomy-angioplasty device 100 and a drive unit 140 operably connected to the atherectomy-angioplasty device 100.

The drive unit 140 includes a drive mechanism 142, specifically, a rotary drive mechanism, configured to rotate the elongate rotor 104 around its longitudinal axis in the rotor lumen 110 of the atherectomy-angioplasty device 100.

The atherectomy-angioplasty system 138 can further include a collection unit 144 fluidly connected to the rotor lumen 110 of the atherectomy-angioplasty device 100, particularly, when the atherectomy-angioplasty device 100 is the rotary device having the atherosclerotic-debris aspiration mechanism set forth above. Such a collection unit 144 is configured for collecting the atherosclerotic debris aspirated into the rotor lumen 110 of the atherectomy-angioplasty device 100 via discharge chamber 146, which is fluidly connected to the collection unit 144 by a conduit 148.

As set forth above, the atherectomy-angioplasty device 100 includes the guidewire lumen 128 along the longitudinal axis of the elongate rotor 104. Accordingly, the atherectomy-angioplasty system 138 can further include a guidewire 150 having a length sufficient to extend through both the atherectomy-angioplasty device 100 and the drive unit 140, as shown. With such a length, the guidewire 150 is configured, at least in part, for advancing the atherectomy-angioplasty device 100 over the guidewire 150 such that the plaque-debulking element 106 is adjacent the atherosclerotic or restenotic plaques in the blood-vessel lumen of the patient.

While not shown, the atherectomy-angioplasty system 138 can further include a fluid-delivery device configured for inflating or deflating the elongate balloon 108 via the inflation lumen 112 with a fluid. Such a fluid-delivery device can include, but is not limited to, a syringe, and the fluid can include saline, optionally, mixed with a radiopaque contrast agent.

### Methods

Methods include at least a method of using the atherectomy-angioplasty system 138. In an example, a method of using the atherectomy-angioplasty system 138 includes obtaining the atherectomy-angioplasty device 100 set forth above. The method also includes advancing the plaque-debulking element 106 of the atherectomy-angioplasty device 100 to a treatment area within a blood-vessel lumen of a patient. The method also includes driving the drive unit 140 operably connected to the atherectomy-angioplasty device 100. As set forth above, the drive unit 140 includes the drive mechanism 142 configured to rotate the elongate rotor 104 around its longitudinal axis in the rotor lumen 110 of the atherectomy-angioplasty device 100 to debulk an atherosclerotic or restenotic plaque in the treatment area. The method also includes collecting atherosclerotic debris from the rotor lumen 110 of the atherectomy-angioplasty device 100 with the collection unit 144 fluidly connected to the rotor lumen 110. The method also includes inflating the elongate balloon 108 via the inflation lumen 112 with fluid from the fluid-delivery device such that the elongate balloon 108 is at least in contact with a blood-vessel wall in the treatment area. Notably, inflating the elongate balloon 108 can be performed before driving the drive unit 140 to debulk the atherosclerotic or restenotic plaque in the blood-vessel lumen of the patient. In such embodiments, the elongate balloon 108 advantageously blocks blood flow by way of its contact with the blood-vessel wall and, thereby, prevents dispersion of atherosclerotic debris from the treatment area. Additionally or alternatively, inflating the elongate balloon 108 can be performed after driving the drive unit 140 to debulk the atherosclerotic or restenotic plaque in the blood-vessel lumen of the patient. In such embodiments, the elongate balloon 108 effectuates angioplasty in the treatment area upon proper placement of the elongate balloon 108. Advantageously, both atherectomy and angioplasty can be effectuated with the atherectomy-angioplasty device 100 in accordance with the foregoing method without exchanging atherectomy and angioplasty devices, thereby reducing the device costs, procedure times, and risks associated with clinician error and patient trauma that accompany separate atherectomy and angioplasty devices.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An atherectomy-angioplasty device, comprising:
a tubular body including a rotor lumen and an inflation lumen;
an elongate rotor disposed in the rotor lumen;
a plaque-debulking element actuated by the elongate rotor when the elongate rotor is rotated around its longitudinal axis in the rotor lumen, the plaque-debulking element configured to debulk atherosclerotic or restenotic plaques in a blood-vessel lumen of a patient; and
an elongate balloon over a distal portion of the tubular body, the elongate balloon fluidly connected to the inflation lumen for inflating or deflating the elongate balloon as desired, optionally, for angioplasty therewith.

2. The atherectomy-angioplasty device of claim 1, wherein the elongate balloon is compactly folded in a ready-to-deploy state of the atherectomy-angioplasty device.

3. The atherectomy-angioplasty device of claim 2, further comprising an expandable stent over the elongate balloon in the ready-to-deploy state of the atherectomy-angioplasty device.

4. The atherectomy-angioplasty device of claim 2, further comprising a retractable sheath over the elongate balloon in the ready-to-deploy state of the atherectomy-angioplasty device.

5. The atherectomy-angioplasty device of claim 4, wherein the elongate balloon includes a coating of an antiproliferative drug on an external surface of the elongate balloon, the retractable sheath protecting the coating prior to retracting the retractable sheath and inflating the elongate balloon for angioplasty therewith.

6. The atherectomy-angioplasty device of claim 1, wherein the elongate rotor approximates a shaftless screw auger that creates a vacuum for aspirating atherosclerotic debris into the rotor lumen when the shaftless screw auger is rotated around its longitudinal axis in the rotor lumen.

7. The atherectomy-angioplasty device of claim 6, wherein the plaque-debulking element is a rotatable cutting tip coupled to the tubular body, the cutting tip coupled to the shaftless screw auger such that the cutting tip rotates around its longitudinal axis for cutting atherosclerotic or restenotic plaques away from the blood-vessel lumen of the patient when the shaftless screw auger is rotated around its longitudinal axis in the rotor lumen.

8. The atherectomy-angioplasty device of claim 7, wherein the cutting tip includes at least one side aperture in fluid communication with the rotor lumen, the side aperture allowing the atherosclerotic debris to be aspirated into the rotor lumen for extracorporeal collection of the atherosclerotic debris.

9. The atherectomy-angioplasty device of claim 6, wherein an absence of a shaft in the shaftless screw auger provides a guidewire lumen for a guidewire.

10. The atherectomy-angioplasty device of claim 1, wherein the elongate rotor approximates a coiled-spring cable.

11. The atherectomy-angioplasty device of claim 10, wherein the plaque-debulking element is an abrasive mass in a distal portion of the coiled-spring cable that orbits around a longitudinal axis of the coiled-spring cable for abrading atherosclerotic or restenotic plaques away from the blood-vessel lumen of the patient when the coiled-spring cable is rotated around its longitudinal axis in the rotor lumen.

12. The atherectomy-angioplasty device of claim 11, wherein the abrasive mass includes an abrasive band around a bump in cable diameter of the coiled-spring cable.

13. The atherectomy-angioplasty device of claim 10, wherein an axial channel through the coiled-spring cable provides a guidewire lumen for a guidewire.

14. An atherectomy-angioplasty system, comprising:
atherectomy-angioplasty device including:
a tubular body including a rotor lumen and an inflation lumen;
an elongate rotor disposed in the rotor lumen;
a plaque-debulking element actuated by the elongate rotor when the elongate rotor is rotated around its longitudinal axis in the rotor lumen, the plaque-debulking element configured to debulk atherosclerotic or restenotic plaques in a blood-vessel lumen of a patient; and
an elongate balloon over a distal portion of the tubular body, the elongate balloon fluidly connected to the inflation lumen for inflating or deflating the elongate balloon as desired, optionally, for angioplasty therewith; and
a drive unit operably connected to the atherectomy-angioplasty device, the drive unit including a drive mechanism configured to rotate the elongate rotor around its longitudinal axis in the rotor lumen of the atherectomy-angioplasty device.

15. The atherectomy-angioplasty system of claim 14, the atherectomy-angioplasty device further comprising an expandable stent over a compactly folded elongate balloon in a ready-to-deploy state of the atherectomy-angioplasty device.

16. The atherectomy-angioplasty system of claim 14, wherein the elongate balloon includes a coating of an antiproliferative drug on an external surface of the elongate balloon.

17. The atherectomy-angioplasty system of claim 14, further comprising a collection unit fluidly connected to the rotor lumen of the atherectomy-angioplasty device, the collection unit configured for collecting atherosclerotic debris aspirated into the rotor lumen.

18. The atherectomy-angioplasty system of claim 14, further comprising:
a guidewire configured for advancing the atherectomy-angioplasty device over the guidewire such that the plaque-debulking element is adjacent the atherosclerotic or restenotic plaques in the blood-vessel lumen of the patient; and
a fluid-delivery device configured for inflating or deflating the elongate balloon via the inflation lumen with a fluid.

19. A method of an atherectomy-angioplasty system, comprising:
obtaining an atherectomy-angioplasty device, the atherectomy-angioplasty device including:
a tubular body including a rotor lumen and an inflation lumen;
an elongate rotor disposed in the rotor lumen;
a plaque-debulking element actuated by the elongate rotor when the elongate rotor is rotated around its longitudinal axis in the rotor lumen; and
an elongate balloon over a distal portion of the tubular body, the elongate balloon fluidly connected to the inflation lumen for inflating or deflating the elongate balloon;
advancing the plaque-debulking element to a treatment area within a blood-vessel lumen of a patient;
driving a drive unit operably connected to the atherectomy-angioplasty device, the drive unit including a drive mechanism configured to rotate the elongate rotor around its longitudinal axis in the rotor lumen of the atherectomy-angioplasty device to debulk an atherosclerotic or restenotic plaque in the treatment area; and
inflating the elongate balloon via the inflation lumen with fluid from a fluid-delivery device such that the elongate balloon is at least in contact with a blood-vessel wall in the treatment area.

20. The method of claim 19, wherein inflating the elongate balloon is performed before driving the drive unit to debulk the atherosclerotic or restenotic plaque in the blood-vessel lumen of the patient, the elongate balloon blocking blood flow by way of its contact with the blood-vessel wall and, thereby, preventing dispersion of atherosclerotic debris from the treatment area.

21. The method of claim 19, wherein inflating the elongate balloon is performed after driving the drive unit to debulk the atherosclerotic or restenotic plaque in the blood-vessel lumen of the patient, the elongate balloon effectuating angioplasty in the treatment area.

22. The method of claim 19, further comprising collecting atherosclerotic debris from the rotor lumen of the atherectomy-angioplasty device with a collection unit fluidly connected to the rotor lumen.
